(19) United States
(12) Patent Application Publication
(10) Pub. No.: US 2024/0188932 A1
(43) Pub. Date: Jun. 13, 2024

(54) ULTRASOUND ON-PROBE VIBRATION SYSTEMS, METHODS AND DEVICES FOR ELASTOGRAPHIC AND VISCOELASTOGRAPHIC MEDICAL IMAGING

(71) Applicant: Elastance Imaging LLC, Columbus, OH (US)

(72) Inventor: Kenneth J. McCaw, Westerville, OH (US)

(73) Assignee: Elastance Imaging LLC, Columbus, OH (US)

(21) Appl. No.: 18/423,241

(22) Filed: Jan. 25, 2024

Related U.S. Application Data
(63) Continuation-in-part of application No. 17/040,824, filed on Sep. 23, 2020.
(60) Provisional application No. 63/488,638, filed on Mar. 6, 2023.
(60) Provisional application No. 63/481,513, filed on Jan. 25, 2023.
(60) Provisional application No. 62/716,303, filed on Aug. 8, 2018.

(51) Int. Cl. A61B 8/08; G01S 7/52

(52) U.S. Cl. CPC A61B 8/485; G01S 7/52042; G01S 7/52079

(57) ABSTRACT

Systems, methods, and devices are provided for inducing acoustic vibration into the human body for the purpose of elastography and viscoelastography medical imaging. This method places the vibration sources on the ultrasound probe separate from the ultrasound array. Compared to ARFI, the methods described herein increase the size and depth of the vibrational shear wave fields and the range of frequencies within the shear wave field and allows for multi-channel and multi-directional audio-frequency vibration sources. Embodiments enable the implementation of various external vibration methods for shear wave elastography and viscoelastography. Additional embodiments enable ARFI push pulses to be generated by the vibration sources, along with the implementation of methods for ARFI elastography and its variants.

### CROSS-REFERENCE TO RELATED APPLICATIONS

The present application is a continuation in part of U.S. patent application Ser. No. 17/040,824 filed Sep. 23, 2020, which in turn claimed priority to International Patent Application Number PCT/US19/23044 filed Mar. 25, 2019, which in turn claimed priority to two U.S. Provisional patent applications: (i) Ser. No. 62/647,672 filed Mar. 24, 2018; and (ii) Ser. No. 62/716,303 filed Aug. 8, 2018, both entitled “SYSTEMS AND METHODS FOR ELASTOGRAPHIC AND VISCOELASTOPGRAPHIC IMAGING”, the disclosures of which are hereby incorporated by reference in their entirety.

The present application additionally claims benefit of priority to two U.S. Provisional patent applications: (i) Ser. No. 63/481,513 filed Jan. 25, 2023; and (ii) Ser. No. 63/488,638 filed Mar. 6, 2023, both entitled “ULTRASOUND ON-PROBE VIBRATION SYSTEMS, METHODS DEVICES AND FOR ELASTOGRAPHIC AND VISCOELASTOPGRAPHIC MEDICAL IMAGING”, the disclosures of which are hereby incorporated by reference in their entirety.

### BACKGROUND

### 1. Technical Field

The invention relates to elastography and viscoelastography devices and to elastography and viscoelastography systems and methods employing an ultrasound probe for ultrasound medical imaging.

### 2. Description of the Related Art

Elastography provides stiffness measurements and maps of a tissue or object, wherein the stiffness values are typically overlaid on an image obtained from a common imaging system such as, but not limited to, ultrasound, magnetic resonance imaging (MRI), computed tomography (CT), and optical coherence tomography (OCT). The stiffnesses are typically obtained by imaging the tissue or object using an imaging or sensing modality that can monitor the propagation of acoustic vibrations that have been injected into or induced in the tissue or object, or by measuring the way the tissue or object deforms or moves in response to an applied force. Elastography has been used in recent times to estimate biomechanical properties such as stiffness of a region of interest (ROI) in a patient, for example, to assess the stiffness of a mass in a breast or abdomen as an aid in deciding if the mass is benign or cancerous or to determine other characteristics of the lesion. Some known techniques include (a) strain or quasistatic elastography, in which the change in shape of a region of interest and its surroundings due to compression is measured; (b) acoustic radiation force impulse (ARFI), in which a focused ultrasound beam momentarily pushes the tissue near the focal point along the beam direction and the resulting tissue displacement and relaxation and/or propagating transverse shear waves are used to measure stiffness within a small (˜3 mm diameter) region of the focal point, along with other related viscoelastic parameters; (c) supersonic shear imaging (SSI), in which ARFI is repeatedly rapidly while quickly increasing the focal point depth to generate an expanding cone-shaped shear wave (a supersonic Mach cone), and the speed of those waves over a large ROI (up to a 5 cm×5 cm sector) is used to measure stiffness and other related viscoelastic parameters; and (d) external vibration shear wave elastography imaging (EV-SWEI), in which external vibrators induce shear waves within the tissue over a large ROI (up to full Doppler range), and the resulting shear waves are used to measure stiffness and other related viscoelastic parameters. MRI, CT, and OCT elastography typically employ some form of EV-SWEI, in which one or more external shakers induce shear waves within the tissue of interest and the shear waves in turn are used to measure stiffness and other related viscoelastic parameters. In one or more embodiments, elastography data may be obtained by Vibration Controlled Transient Elastography (VCTE) also known as FIBROSAN® (Echosens, Paris, France), or by Acoustic Radiation Force Impulse (ARFI), Supersonic Shear Imaging (SSI) elastometry, or any other elastography technique. As used herein, the term “ARFI” shall mean Acoustic Radiation Force Impulse (ARFI), and its variants including SSI.

The conventional ARFI technique utilizes a single transducer for both transmitting the radiation force and tracking the resulting displacement of tissue. To obtain displacement information, a brief acoustic radiation force (0.003-1 ms) is transmitted to a focal point within the ROI to generate a localized displacement in tissue. Immediately thereafter, Doppler ultrasound is acquired for monitoring peak displacement and recovery of the tissue for approximately 4-6 ms. By repeating this push-track procedure at multiple focal points within the ROI, a 2D stiffness (elastography) image can be created. Because of its ease of implementation, ARFI is currently included as a modality in multiple commercial ultrasound systems. However, now that ARFI elastography is widely available, problems are starting to emerge that limit its utility.

ARFI uses focused, short-duration high-intensity push pulses, produced by the same crystals that provide the imaging results, in a push-track, push-track, push-track, etc. pattern. The pushes produce tissue displacement up to 10 microns though usually less. The degree of displacement is related to tissue stiffness; softer tissues will displace more than stiffer tissues. However, because the push pulse requires stimulation of the embedded ultrasound probe crystals with more voltage than is required for standard B-mode or Doppler ultrasound imaging, the crystals may heat up to unusable levels, degrading or even destroying the probe, and the corresponding energy intensity produced at the skin surface and at the beam's focal point may also cause tissue damage. Therefore, under regulatory safety pressure, manufacturers generally limit the use of ARFI to 8 cm depth, though current international elastography guidelines discourage depths more than 6 cm due to reduced accuracy. These limitations make ARFI unusable for deeper tissues within the body and especially impacts its use in obese patients. For usage guidelines, sec for example, G. Ferraioli, et al. (2018), “Liver Ultrasound Elastography: An Update to the World Federation for Ultrasound in Medicine and Biology Guidelines and Recommendations”, Ultrasound in Medicine & Biology, Vol. 44(12), 2018, Pp 2419-2440, ISSN 0301-5629 (doi: 10.1016/j.ultrasmedbio.2018.07.008). Concerningly, ARFI has been shown to rupture rat lung capillaries due to its high pulse energies reverberating at tissue-air boundaries (see D. L. Miller, ct al. (2019), “Pulmonary Capillary Hemorrhage Induced by ARFI Shear Wave Elastography in Ventilated Rats”, J Ultrasound Med. 2019; 38(10):2575-258 (doi: 10.1002/jum.14950).

Curvilinear probes are recommended for liver elastography, the most common type of abdominal elastography, because the imaging depths are typically more than a few centimeters. However, a problem arises with ARFI when scanning more than a few centimeters deep. Because the focus and sharpness of the ARFI push pulse rapidly loses bandwidth with increasing depth, the highest shear wave frequency within the group of shear waves produced at a focal point more than a few centimeters deep will generally be less than 200 Hz. This is very limiting, not only in terms of spatial resolution, but also in ARFI's ability to estimate viscoelastic parameters such as dispersion. To clarify and illustrate why this is so limiting, consider the following. In the field of elastography, objects with a diameter of approximately ¼ the length of an interrogating shear wave are just at the edge of detectability. However, any noise in the data (and ultrasound is noisy), feathering of the object edges, or sudden tissue impedance changes at the object borders, can render the object undetectable. Therefore, it is far preferable for the shear waves interrogating such an object to have wavelengths the same size as, or smaller than, the object. Wavelength decreases with increasing frequency, so in general, higher frequencies will enable detection of smaller objects. As a specific example, consider a 2 mm diameter soft tissue mass with 10 m/s stiffness (with a speed of sound of 1540 m/2). Such a mass would require at shear wave frequency of at least 125 Hz for ¼ wave to detect it under perfect conditions. Since ultrasound is not perfect, a more preferable shear wave would have a wavelength of 2 mm or less, or a frequency of 500 Hz or more. For assessment of the mass's viscoelastic properties such as dispersion, even higher shear wave frequencies would be required, such as 1000 Hz or more, to provide any kind of confidence in the estimates. Thus, with only modest depth (a few centimeters), ARFI dispersion measurements become unreliable and ARFI stiffness measurements rapidly lose spatial resolution, supporting and perhaps explaining the 6 cm limit within the clinical guidelines.

All these issues are further compounded when the ultrasound probe face is not positioned parallel to the organ capsule surrounding the ROI. In these instances, the ARFI pulse can lose so much power that the tissue force becomes too weak to produce reliable stiffness measurements. See, for example, FIG. 5.1 in Foundation for the National Institutes of Health Biomarkers Consortium, Non-Invasive Biomarkers of MetaBolic Liver DiscasE (NIMBLE) Project Steering Committee, “Biomarker Qualification Letter of Intent (LOI) Content Elements” (addressed to the FDA), Dec. 10, 2020. Hence, current elastography guidelines recommend parallel placement of the probe face to organ capsules (see Table 2 in G Ferraioli, et al., 2018).

In summary, the problems related to ARFI include: (a) it is generally depth-limited in human patients to less than 7 cm depth, which impairs the scanning of organs and tissues deep within the body, such as liver, kidney, pancreas, heart, etc., especially in obese patients; (b) it can be destructive to tissues; (c) it is destructive to ultrasound probes, so repair or replacement may be required every few years; and (d) ARFI pulses that are off-axis to an organ's fibrous capsule (and other high acoustic impedance boundaries) severely degrade the reliability of tissue stiffness measurements.

### SUMMARY OF THE INVENTION

In one aspect, the present disclosure provides an ultrasound elastography diagnostic apparatus including a probe assembly that includes an ultrasound probe configured to detect vibrations passing through subject tissue. The probe assembly includes a vibration isolation component. The probe assembly includes one or more vibratory devices coupled to the ultrasound probe via the vibration isolation component. The probe assembly includes an input electrical interface communicatively couplable to a signal generator to receive a vibration driver signal comprising one of: (i) an acoustic radiation force impulse (ARFI) signal; and (ii) an external vibration shear wave elastography imaging (EV-SWEI) signal. The input electrical interface is communicatively coupled to the one or more vibratory devices to generate a corresponding one of an ARFI push pulse and an EV-SWEI vibration. The probe assembly includes an output electrical interface communicatively coupled to the ultrasound probe that receives a tracked vibration waveform. The output electrical interface is communicatively couplable to an image processor to generate a map of one or more viscoelastic properties contained in the tracked vibration waveform.

In another aspect, the present disclosure provides a method for making an ultrasound elastography diagnostic apparatus. In one or more embodiments, the method includes attaching one or more vibratory devices to a vibration isolation component. The method includes forming a probe assembly by attaching the vibration isolation component to an ultrasound probe configured to detect vibrations passing through subject tissue. The method includes communicatively coupling an input electrical interface to the one or more vibratory devices that are configured to generate at least one of (i) an acoustic radiation force impulse (ARFI) push pulse; and (ii) an external vibration shear wave elastography imaging (EV-SWEI) vibration. The method includes communicatively coupling an output electrical interface to the ultrasound probe.

In an additional aspect, the present disclosure provides a method of performing an ultrasound elastography diagnostic procedure. In one or more embodiments, the method includes positioning a probe assembly into contact with subject tissue. The probe assembly includes: (i) an ultrasound probe configured to detect vibrations passing through the subject tissue; (ii) a vibration isolation component; and (iii) one or more vibratory devices coupled to the ultrasound probe via the vibration isolation component and comprising one or more vibratory devices that are configured to generate at least one of (a) an acoustic radiation force impulse (ARFI) push pulse; and (b) an external vibration shear wave elastography imaging (EV-SWEI) vibration. The method includes transmitting one of an ARFI signal and an EV-SWEI signal to the one or more vibratory devices to produce a corresponding one of the ARFI push pulse and the EV-SWEI vibration. The method includes detecting, at the ultrasound probe, a tracked vibration waveform resulting from the corresponding one of the ARFI push pulse and the EV-SWEI vibration. The method includes mapping, at an image processor, one or more viscoelastic properties derived from the tracked vibration waveform that results.

These and other features are explained more fully in the embodiments illustrated below. It should be understood that in general the features of one embodiment also may be used in combination with features of another embodiment and that the embodiments are not intended to limit the scope of the invention.

### BRIEF DESCRIPTION OF THE DRAWINGS

The description of the illustrative embodiments can be read in conjunction with the accompanying figures. It will be appreciated that for simplicity and clarity of illustration, elements illustrated in the figures have not necessarily been drawn to scale. For example, the dimensions of some of the elements are exaggerated relative to other elements. Embodiments incorporating teachings of the present disclosure are shown and described with respect to the figures presented herein, in which:

FIG. 1 is a view of dual piezo transducer bars attached to standard ultrasound probe, providing mono or stereo full range audio-frequency acoustic vibrational fields;

FIG. 2 is a view of segmented piezo transducer bars attached to standard ultrasound probe, providing multi-channel full range audio-frequency acoustic vibrational fields or ultrasound-frequency ARFI push pulses;

FIG. 3 is a view of front face (patient contact surface) showing positions of piezo transducer bars related to ultrasound probe array;

FIG. 4 is a view of front faces (patient contact surface) of three common ultrasound types showing positions of piezo transducer bars related to ultrasound probe arrays;

FIG. 5 is a view of acoustic vibrations patterns using an EV-SWEI method with dual piezo bars;

FIG. 6 is a view piezo transducer bar assembly to be installed permanently into ultrasound probe housings (end view);

FIG. 7 is a view piezo transducer bar installed permanently into ultrasound probe housings (front face view);

FIG. 8 is a flow diagram of the electronic system used to send signals to the device(s);

FIG. 9 is a view of two tissue contacting piezo tactile driver rings or half rings embedded into an endocavity ultrasound probe;

FIG. 10 is a view of a tissue contacting piezo tactile drivers embedded into an endocavity ultrasound probe;

FIG. 11 is a view of a multi-source/multi-channel piezo tactile driver buttons or rod tips surrounding an ultrasound linear probe;

FIG. 12 is a block diagram of an ultrasound elastography diagnostic apparatus that includes a probe assembly that includes an ultrasound probe and vibratory device(s) communicatively coupled to a signal generator and an image processor,

FIG. 13 is a flow diagram of a method for making an ultrasound elastography diagnostic apparatus; and

FIG. 14 is a flow diagram of a method of performing an ultrasound elastography diagnostic procedure.

### DETAILED DESCRIPTION

The systems and methods described herein overcome the ARFI problems by enabling: (a) the construction of more durable probes for ARFI elastography; (b) the spreading the ARFI push-pulse sources over a greater surface area to enable deeper penetration with less risk of damage to surface tissues; and (c) the utilization of EV-SWEI methods without the need for additional external vibrators (though additional external vibrators can be used if desired to enable very deep shear wave penetration into tissues). The ability to utilize a single probe without additional external shakers and without overdriving the tracking elements provides an attractive, convenient package for performing either ARFI or EV-SWEI. Furthermore, by switching to EV-SWEI, safety concerns related to ARFI's push-pulse ultrasound intensity are eliminated both at the surface and at the focal point, and in addition, the depth limitations associated with ARFI are eliminated. A further advantage of utilizing EV-SWEI as described herein is the ability to generate shear wave frequencies spanning a far greater range than ARFI, exceeding 5,000 Hz. This larger range not only improves image resolution because of the smaller wavelengths associated with the higher frequencies, but it also increases the reliability of measures of viscoelasticity. Taken together, the improvements provided by the systems and methods described herein enable better characterization of tissue mechanical properties, and thereby are better at finding the smallest cancer masses and other abnormalities.

The method described herein separates the push-track functions of ARFI into two separate devices, isolated with shock dampening material, and places the push into a separate apparatus surrounding the ultrasound probe. This system allows for greater control of vibrations including greater acoustic range and depth penetration into tissue, and avoids heating the ultrasound probe. It also allows for continuous vibration as in EV-SWEI, which provides multiple advantages as described above. The ultrasound probe also does not have to switch back and forth between push and track, which allows for more complete and thorough real-time imaging, and gating of moving tissue such as the heart.

In one aspect, the present disclosure provides systems, methods, and apparatuses (hereinafter the “system”) that include monitoring a signal from a set of sensors placed on a user.

Disclosed are systems, methods and devices designed to provide continuous high-fidelity, full range (5-8,000 Hz), simultaneous multifrequency vibrations within the human body, with the waveforms remaining consistent to the externally-induced waveforms. Frequencies in the upper ranges are necessary to achieve fine specificity of tissue targets, for example, 1,000-6,000 Hz in cornea, 40-1,200 Hz in liver, 40-3,000 Hz in breast, etc. The full frequency range allows the user to obtain maximum specificity by removing limitations imposed by inferior vibration systems, so that the maximum upper frequency can now be selected based on the round-trip travel time needed for ultrasound tracking of tissue displacement, the size of the smallest object to be detected, and the highest anticipated stiffness within the tissue.

Thus, in one aspect, the present disclosure provides systems, methods, and apparatuses for external vibration elastography and viscoelastography (EV-SWEI). In another aspect, the present disclosure provides systems, methods, and apparatuses for acoustic radiation force impulse (ARFI) ultrasound elastography and viscoelastography.

The systems of the present invention are able to provide the following advantages over the known ARFI systems: (a) lessened risk of damaging the ultrasound transducer elements (the piezoelectric material for transmitting and receiving ultrasound); (b) lessened risk of damaging patient tissues; (c) a wider frequency range of up to 5000 Hz or more, which leads to better resolution and better viscosity measurement of small masses; (d) availability of multiple sources of waves, which can produce crawling waves, reverberant waves, etc., which provide for extended depth of imaging and wherein several independent signals produced at the same time are capable of providing for stiffness and viscosity evaluation, which means that the system will be better able to detect smaller masses while improving characterization of the tissues to help differentiate healthy tissue from carcinoma, benign tumor, etc.

With existing ultrasound imaging, some cancers and other anomalies are difficult to detect in deep tissue while in their smallest and earliest stages of development. The disclosed method provides a solution for the detection of small cancer masses and other anomalies deep within the human body often without the need for an invasive elastography procedure. This method is non-destructive to hardware or tissue, and is non-invasive to the subject (patient).

The present innovation provides an acoustic radiation force impulse (ARFI) ultrasound diagnostic apparatus wherein the two functions of ARFI are divided into two separate hardware systems, while both remain enclosed in the same apparatus (probe). The present innovation also provides for methods of using an acoustic radiation force impulse (ARFI) system wherein the two functions of ARFI are divided into two separate hardware systems, while both remain enclosed in the same apparatus (probe). Typically, a subject will be a human desiring an assessment because a clinician wishes to use the invention as a part of a diagnosis. Systems or sets with more than two probes can also be used, so that, in one embodiment, the methods can include an ultrasound system to concurrently process first signals from the first ultrasound probe and second signals from the second ultrasound probe. A probe can be adapted for a particular anatomical region or indication. For example, the anatomical region can be selected from the group consisting of the forehead region, anterior tibia region, foot region, distal radius region, elbow region, presternal region, temporal bone region, as well as regions requiring endocavity probes such as vaginal, rectal, esophageal, etc., regions.

According to some embodiments, dual piezo transducer bars are attached to a standard ultrasound probe, providing mono or stereo full range audio-frequency acoustic vibrational fields in a system for estimating and displaying an internal region of a subject comprising: a source of shear waves propagating in multiple directions, the source being configured to concurrently induce shear waves at respective different vibration frequencies in a region of interest (ROI) in the subject; an imaging system measuring displacement as a function of time of respective voxels in the ROI in the presence of the induced shear waves; a computer processor configured to apply computer algorithms to the displacements and account for the vibration frequencies to calculate respective shear wave speeds in the ROI and to further calculate a respective internal display.

According to some embodiments: the processor for the dual piezo audio-frequency transducer system can be further configured to account in the computer algorithms for effects of attenuation (alpha) of the shear waves as they propagate, the source of shear waves can comprise a surface with plural sources of vibration frequencies embedded in an active region of the surface, wherein the plural sources are configured to vibrate concurrently; the imaging system can comprise an ultrasound scanner and an imaging ultrasound transducer; the scanner and transducer can be configured to measure the displacements to a depth in the patient of at least 10 cm, and to measure the displacement in a patient's liver or in the patient's breast; the imaging system can be an MRI scanner or an OCT (Optical Coherence Tomography) scanner rather than an ultrasound scanner; the vibration frequencies can include at least frequencies up to 5000 Hz, and can include frequencies in the range of 40-7000 Hz; and the source can be configured to step the vibration frequencies is selected steps within a selected range of frequencies.

According to some embodiments, the dual piezo audio-frequency transducer is made from one of a piezoceramic material, a piezoceramic composite material, a piezoceramic single-crystal material, a capacitive micro electromechanical ultrasonic transducer chip, a piezoceramic micro electromechanical ultrasonic transducer chip, or a polymeric piezo material. According to some embodiments, the dual piezo audio-frequency transducer is an array made from one of a piezoceramic material, a piezoceramic composite material, a piezoceramic single-crystal material, a capacitive micro electromechanical ultrasonic transducer chip, a piezoceramic micro electromechanical ultrasonic transducer chip, or a polymeric piezo material. In one case, the center element array and the lateral element arrays are capacitive micro electric mechanical ultrasonic transducers (CMUT). In another case, the center element array and the lateral element arrays are piezoceramic micro electromechanical ultrasonic transducers (PMUT).

The invention relates to diagnostic medical imaging, and more particularly relates to elastography. In its most immediate sense, the invention relates to ultrasound elastography, as well as to magnetic resonance elastography and to optical coherence elastography. Some soft tissue diseases (including but not limited to some cancers) cause the diseased region to have viscoelastic properties that are different from those of the surrounding non-diseased tissue. For example, a breast tumor may be stiffer than the surrounding healthy breast tissue. As used herein, “elastography”, we mean both elastography and viscoelastography. Technically, viscoelastography includes both stiffness and dispersion (and/or viscosity), whereas technically elastography includes only stiffness. The present disclosure recognizes that common usage in the field has changed, such that “elastography” usually includes both stiffness and dispersion (and/or viscosity) but sometimes elastography is still used to mean only stiffness. The present disclosure may apply to improve commercial machines with elastography that also estimate dispersion (and/or viscosity).

Ultrasonic elastography can be used in vivo to localize diseased regions within an organ. In an ultrasonic elastography study, shear-inducing transducers acting as vibration sources introduce acoustic energy into the organ of interest, generating shear waves within the organ and causing the organ tissue to distort. By localizing the regions where the distortion changes and measuring the distortion of those regions, various material properties can be determined, such as stiffness, elasticity, viscosity, attenuation, wave speed, phase angle, and frequency dispersion, so that the locations and severities of the disease can be determined.

One type of ultrasound elastography utilizes a reverberant field (also known as a reverberant shear wave field). In this technique, one or more shear-inducing transducers are used to create a profusion of shear waves along many different directions. This diversity of differently-directed shear waves is also enhanced by all the reflections that naturally occur from the boundaries of the organ and from the inhomogeneities within it.

It is possible to estimate shear wave speed (“SWS”) and soft tissue stiffness and then the soft tissue dispersion which is the SWS change with frequency and is related to the lossy and attenuating nature of tissue. However, in the past such estimations required the use of autocorrelation with a wide autocorrelation window. This could limit the spatial resolution of the shear wave speed map that indicates the position of a suspect structure in the organ under examination. Also, there are conditions where the shear wave field may not be uniformly reverberant. Such conditions are created, for example, in locations close to a strong vibration source producing vibrations only in one direction, or when slip surfaces surrounding certain organs such as the heart allow vibrations to enter the organ only from certain connecting tissues, or when anatomy creates a region that naturally focuses and reinforces certain wavelengths from only certain directions while blocking others, such as the pelvis.

The family of invasive ultrasonic imaging probes includes various shapes and designs adapted to fit the internal morphology of the organ to be imaged. A distinction can be made between (i) endocavity probes which are used for endo-vaginal and endo-rectal diagnostics, (ii) endoscopic probes that are elongated versions of invasive instruments wherein the imaging transducer is mounted at the extremity of the (flexible or rigid) tube of an endoscope, which is, in turn, attached to an endoscope handle on which the control functions for the instruments are typically provided, (iii) catheter based probes wherein the ultrasonic transducer is mounted at the extremity or distal end of the corresponding catheter tube, and (iv) special imaging devices designed for specific applications such as brain imaging (e.g., a “Burr Hole” probe) or surgical monitoring (e.g., “Per-Op” probes). Generally speaking, catheter-based instruments for ultrasonic diagnostics are very similar to endoscopic tubes but have a much smaller tube diameter, while Burr Hole type probes are considered to be a customized version of endocavity probe devices. Surgical monitoring (Per-Op) probes are specialized instruments that are specifically designed to fit each particular surgical application. Accordingly, there is a large variety of such instruments, with a small housing being a common characteristic thereof.

The above summary contains simplifications, generalizations and omissions of detail and is not intended as a comprehensive description of the claimed subject matter but, rather, is intended to provide a brief overview of some of the functionality associated therewith. Other systems, methods, functionality, features and advantages of the claimed subject matter will be or will become apparent to one with skill in the art upon examination of the following figures and detailed written description.

Definitions: Embodiments described below in context of the user input devices are analogously valid for the respective methods, and vice versa. Furthermore, it will be understood that the embodiments described below may be combined, for example, a part of one embodiment may be combined with a part of another embodiment.

It should be understood that the terms “on”, “over”, “top”, “bottom”, “down”, “side”, “back”, “left”, “right”, “front”, “lateral”, “side”, “up”, “down” etc., when used in the following description are used for convenience and to aid understanding of relative positions or directions, and not intended to limit the orientation of any device, or structure or any part of any device or structure. In addition, the singular terms “a”, “an”, and “the” include plural references unless context clearly indicates otherwise. Similarly, the word “or” is intended to include “and” unless the context clearly indicates otherwise.

Acoustic communication refers to the passage of sound waves between two points in a predetermined manner. Usually, this is accomplished by selecting a desired pathway between the two points that permits the passage of sound waves either directly or indirectly. For ultrasound, direct passage of ultrasound waves would occur, for instance, when an ultrasound crystal is directly disposed to (usually touching) an acoustic coupling material, such as a composite. Indirect passage of ultrasound waves would occur, for instance, when an ultrasound crystal is located at a predetermined distance from an acoustic coupling material or when a number of acoustic coupling materials, often heterogenous materials, form two or more layers.

Acoustic coupler for ultrasound refers to a connection or plurality of connections between an ultrasound crystal and a substance that reflects or passes ultrasound pulses and is not part of the device. The acoustic coupler will permit passage of ultrasound waves. It is desirable for such couplers to minimize attenuation of ultrasound pulses or signals and to minimize changes in the physical properties of an ultrasound wave, such as wave amplitude, frequency, shape and wavelength.

Crystal refers to the material used in the ultrasound transducer to transmit ultrasound waves and includes any current and future material used for this purpose. Crystals typically consist of lead zirconate titanate, barium lead titanate, lead metaniobate, lithium sulfate and polyvinylidene fluoride or a combination thereof. A crystal is typically a piezoelectric material, but any material that will contract and expand when an external voltage is applied can be used, if such a material can generate ultrasound waves described herein and known in the art. Crystals emit ultrasound waves because the rapid mechanical contraction and expansion of the material moves the medium to generate ultrasound waves.

Detector refers to any structure capable of measuring an ultrasound wave or pulse, currently known or developed in the future. Crystals containing dipoles are typically used to measure ultrasound waves. Crystals, such as piezoelectric crystals, shift in dipole orientation in response to an applied electric current. If the applied electric current fluctuates, the crystals vibrate to cause an ultrasound wave in a medium. Conversely, crystals vibrate in response to an ultrasound wave that mechanically deforms the crystals, which changes dipole alignment within the crystal. This, in turn, changes the charge distribution to generate an electric current across a crystal's surface. Electrodes connected to electronic circuitry sense a potential difference across the crystal in relation to the incident mechanical pressure.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker). The terms “individual,” “patient,” or “subject” encompass mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In some embodiments, the mammal is a human.

Linear array refers to a transducer design where the piezo elements are arranged in a linear fashion along one or more axes. The elements can be fired in sequential, as well as non-sequential and simultaneous firing patterns or a combination thereof. With sequential firing, various beams can be formed based on the sequence, the intensity, and the delays between elements. The number of elements in one array usually determines the fineness with which a beam can be steered. With segmental firing, a group or segment of elements can be activated simultaneously resulting in a deeper near field and a less divergent far field compared with sequential activation. A segmental linear array produces, however, courser beams when compared to a sequential linear array with the same number of elements. As used herein, the term “piezo element” refers to the piezo elements within a standard ultrasound probe for clinical diagnostic imaging (as context indicates). As used herein, the terms “piezo bar”, “piezo transducer” and “piezo button” refer to the additional “snap on” or “built in” piezo elements that provide the separate function for producing ARFI push pulses and EV-SWEI vibrations, though these are sometimes referred to as “piezo elements” where the context is clear.

Transmission angle refers to the angle of an ultrasound beam that intersects the object or tissue plane. The transmission angle is normally measured with respect to the object or tissue plane. The object or tissue plane has a reference angle of zero degrees.

Transmission frequency refers to the frequency of the wave that is being transmitted from the source. Transmission frequency for medical ultrasound ARFI typically ranges between 1 and 8 MHz. Higher frequencies usually provide higher spatial resolution, but tissue penetration decreases with higher frequencies, especially in dense fat tissue. Lower transmission frequencies are generally characterized by lower spatial resolution but with improved tissue penetration. Transmission frequencies for EV-SWEI typically range between 40 and 5,000 Hz and have much longer wavelengths than ultrasound waves, and so they can penetrate deeply. They are less directional, but can still be partially steered, especially once they convert to shorter wavelength shear waves. Furthermore, at these low frequencies, it is straightforward to combine multiple frequencies to form a complex waveform for the transmission. The EV-SWEI transmission frequencies within this disclosure are generally called “audio-frequencies” since the audio frequency range generally matches the EV-SWEI frequency range.

Ultrasound pulse refers to any ultrasound wave transmitted by an ultrasound source. Typically, the pulse will have a predetermined amplitude, frequency, and wave shape. Ultrasound pulses may consist of sine waves with single frequency or varying frequencies, as well as single amplitudes and varying amplitudes. In addition to sine waves, square waves or any other wave pattern may be employed. Square waves may be obtained by adding single-frequency sine waves to other sine waves. The summation of waves can then result in a square wave pattern. Similarly, audio-frequency waves may be combined with similar results for short pulses as well as for continuous waveforms.

Ultrasound signal refers to any ultrasound wave measured by an ultrasound detector after it has been reflected from the interface of an object or tissue. Clinical ultrasound signals typically range in frequency between 1 and 35 MHz.

Ultrasound source refers to any structure capable of generating an ultrasound wave or pulse, currently known or developed in the future. Crystals, such as piezoelectric crystals, that vibrate in response to an electric current applied to the crystal can be used as an ultrasound source. The source may be made from one of a piezoceramic material, a piezoceramic composite material, a piezoceramic single-crystal material, a capacitive micro electromechanical ultrasonic transducer chip or a piezoceramic micro electromechanical ultrasonic transducer chip. PZT (lead zirconate titanate) is used often as the material of the transmission piezoelectric element, however the materials not containing lead may also be used. These include rock crystal, lithium niobate (LiNbO3), niobic acid tantalic acid potassium [K(Ta,Nb)O3], barium titanate (BaTiO3), lithium tantalate (LiTaO3) and strontium titanate (SrTiO3). Similarly, audio-frequency source refers to any structure capable of generating an audio-frequency wave or pulse, currently known or developed in the future. The materials used to make audio-frequency sources have been disclosed earlier. In some embodiments, the audio-frequency source is a piezo audio-frequency transducer (or piezo tactile transducer) made from the piezo materials used in audio loudspeakers for high frequency or “tweeter” drivers. In some embodiments, the source is a piezoceramic audio-frequency transducer (or piezoceramic tactile transducer). In yet other embodiments, the audio-frequency source is the same as an ultrasound source so it can be configured to emit either ARFI frequency push pulses or audio-frequency vibrations.

The term “ultrasound transducer” and “ultrasound probe,” as used herein, are used interchangeably.

Ultrasound wave refers to either an ultrasound signal or pulse.

In one or more embodiments, the present innovation provides an acoustic radiation force impulse (ARFI) ultrasound diagnostic apparatus wherein the two functions of ARFI are divided into two separate hardware systems, while both remain enclosed in the same apparatus (probe). The present innovation also provides for methods of using an acoustic radiation force impulse (ARFI) system wherein the two functions of ARFI are divided into two separate hardware systems, while both remain enclosed in the same apparatus (probe). In one or more embodiments, the two functions are divided into two separate hardware systems but not combined with the same probe (e.g., a snap-on boot that goes around an ultrasound probe).

Audio frequency tactile vibration is produced by sound waves that are transferred through mass rather than through the air, by means of physical vibration. Elastography is a method of medical imaging that applies audio frequency tactile vibrations to the human body and then, using a standard method of imaging such as, but not limited to, ultrasound, measurements of how the tissue reacts to various vibrational waveforms and audio frequencies are used to determine stiffness and viscosity of the tissue. Cancerous lesions, for example, are generally harder than surrounding healthy tissue, allowing the system to detect and map the size, shape and characteristics of the cancer. This method is also used for determining scar tissue, fat, steatosis, cirrhosis, inflammation, and many other abnormalities.

Acoustic radiation force impulse (ARFI) is a common type of elastography in which the tactile vibration force is produced by an ultrasound array, and not by using an external vibration source, or a source other than the array itself. This method has inherent risks both to the safety of the patient and to the durability of the ultrasound probe.

ARFI requires a short power surge to be injected into the ultrasound array crystals causing the crystals to react to the surge with a momentary increase is physical size. This causes a short burst of tactile vibration which is then focused into an area typically within 6.5 cm of the ultrasound contact point. The ultrasound probe function is then instantly switched to receive mode and the vibrational effect in the form of shear waves produced by the burst of energy in the tissue is mapped, providing stiffness numbers. However, the sudden burst of energy at the focal point may damage the tissue. The burst of energy in the probe array can also cause damage to the ultrasound crystals. Probes used for ARFI elastography are routinely used only for only a few years and then must be replaced or repaired.

In one or more embodiments, the endocavity ultrasound probe may be a linear probe, a sector probe, a convex probe, or other configuration of endocavity probe. In one or more embodiments, the ultrasound probe may have one or more piezoelectric transducers (vibrators) for transmitting ultrasound waves and one or more piezoelectric transducers (vibrators) for receiving ultrasound waves.

The present invention may be introduced into a person's anatomy via, for example, a natural orifice or by percutaneous or surgical access to a lumen, vessel, or body cavity. It should be understood that, although the present system and method will be described in connection with percutaneous cardiac intervention of a person, the percutaneous or surgical intervention and access may be to any percutaneous intervention of any biological being, such as animals, or to non-biological objects such as to probe devices (e.g., electronic devices, inanimate objects, etc.) or structures (e.g., buildings, caves, etc.) through small openings. Further, the present system is also applicable to other forms of Doppler sonography. Further, although embodiments are described related to an endocavity probe, the present systems, devices and methods are equally applicable to any endoscopic device for imaging, inserted through any orifice, such as, transnasal, transvaginal, transrectal, transesophageal echocardiogram (TEE) probe, endocavity probes, etc.

One embodiment of the endoscopic device for imaging is a transesophageal echocardiogram (TEE) probe for insertion into an esophagus, where such a TEE probe is used for describing the present devices, systems and methods. However, it should be understood that any other type of probe may be used in any desired surgical and imaging applications such as for insertion into any bodily orifice, such as the throat, nose, rectum, etc. As used herein, an “endocavity probe” includes transrectal, transvaginal, and other endocavity probes such as a transesophageal (TEE) probe. Endocavity probes can be used for prostate, pelvic floor and urethra scanning and imaging. As used herein, an “endocavity ultrasound” includes endorectal ultrasound (ERUS) and transrectal ultrasound (TRUS) embodiments.

Further, the inventive endoscopic devices for imaging according to the present devices, systems and methods may be used alone or in conjunction with surgical instrument for performing desired surgery, such as removal or destruction of undesired growth or tissue, etc. The inventive endoscopic devices may be used for non-invasive or minimally invasive procedures for therapeutic and imaging purposes, and may be self-guided, such as automatically and/or manually e.g., using a joystick, or guided using any conventional guiding devices.

Generally, ARFI only generates a frequency range below 200 Hz, unless ARFI is used close to the surface (<3 cm). However, extended frequency ranges are necessary for determining the smallest target masses, and for determining the dispersion (viscosity) of the target mass.

The proposed method herein causes no damage to the ultrasound hardware. And because the vibrational effect is spread out over a much wider area, the damage to human tissue is minimized greatly. In one or more embodiments, the frequency range is limited to the range possible with piezo audio-frequency transducer materials, currently 5 to 20,000 Hz. And because there is no need to switch the ultrasound probe from push to track, the ultrasound probe can remain in its normal contact function, while the vibrational sources can also remain in continual function, allowing the system to improve greatly.

In one or more embodiments, the system includes a power supply configured to apply the proper power to the transducer to image tissues within a patient. For example, the power input into the transducer might be 150 W, 200 W, 500 W 750 W, or 1000 W to achieve output suitable for deep imaging in a patient.

The ultrasound transducer may be, for example, any one of a magnetostrictive ultrasound transducer using a magnetostrictive effect of a magnetic body, a piezoelectric ultrasound transducer using a piezoelectric effect of a piezoelectric material, and a capacitive micromachined ultrasound transducer (cMUT), which transmits and receives ultrasound waves using vibration of several hundred or several thousands of micromachined thin films. In addition, other kinds of transducers that generate ultrasound waves according to an electrical signal or generate an electrical signal according to ultrasound waves may also be used as the ultrasound transducer.

For example, the ultrasound transducer element may include a piezoelectric vibrator or a thin film. When alternating current is applied to piezoelectric vibrators or thin films of the ultrasound transducers from a power source, the piezoelectric vibrators or thin films vibrate with a predetermined frequency according to applied alternating current and ultrasound waves of the predetermined frequency are generated according to the vibration frequency. On the other hand, when ultrasound echo waves of the predetermined frequency reach the piezoelectric vibrators or thin films, the piezoelectric vibrators or thin films vibrate according to the ultrasound echo waves. In this regard, the piezoelectric vibrators or thin films output alternating current of a frequency corresponding to the vibration frequency thereof.

In one or more embodiments, the method proposed herein is to take the two functions of the ultrasound probe currently found in ARFI, and separate the two functions into two separate hardware systems, while both remain enclosed in the same apparatus (probe). In one or more embodiments, the method allows for the benefits of ARFI to be realized without damage to hardware or tissue, and with the added benefits of much greater frequency range, much wider fields of shear wave vibration and continual operation for both probe and vibration source.

In one or more embodiments, the ultrasound system of the present invention uses acoustic radiation force impulse (ARFI). In one or more embodiments, the ultrasound system uses other ultrasound modalities such as external vibration shear wave elastography imaging (EV-SWEI), color-flow, B-mode, A-mode, M-mode, spectral Doppler, acoustic streaming, tissue Doppler module, and C-scan.

In one or more embodiments, the method proposed herein separates the ARFI pulse push function from the ultrasound probe and places it into bars made of piezo material similar to the crystals used in ultrasound arrays. This type of piezo material is commonly used in audio loudspeakers, generally as high frequency or “tweeter” drivers. In one or more embodiments, the bars are sealed in glass or polycarbonate sleeves, which isolates the piezo material from tissue, and then mounted into rubber isolation material which limits vibration from “feeding back” into the ultrasound probe. In one or more embodiments, the vibration assembly may be mounted into a removable snap-on rubber boot, molded to fit a multitude specific probe designs. In one or more embodiments, the vibration assembly may also be built directly into probes and manufactured by commercial ultrasound hardware manufacturers.

In one or more embodiments, only two piezo sources are provided for two channel (stereo). In other embodiments, segmented piezo sources allow for multichannel (surround). In yet other embodiments, the number of piezo sources on each side of the ultrasound transducer matches the number of ultrasound elements, while in yet other embodiments, the number may exceed the number of ultrasound elements. For embodiments targeting EV-SWEI methods, at least two independent sources are positioned on either side of the ultrasound array to allow for unique elastography modalities such as crawling wave, reverberant and many types of sectional phase inversion, delays, and other techniques known to those skilled in the field. Each modality may provide unique results beyond the capabilities of current ARFI systems. Thus, the system improves all aspects of elastography and viscoelastography imaging modalities.

In one or more embodiments, the piezo vibration assembly is powered by amplifiers matched to the impedance characteristics of the piezo bar materials. Audio frequency waveforms, which are designed specifically for types of tissue, body sizes, BMI, etc. are produced by software within the ultrasound machine and sent to the amplifiers. In one or more embodiments, the imaging system turns the vibration on, then the ultrasound elements within the probe are used to take the scan, then both are immediately turned off.

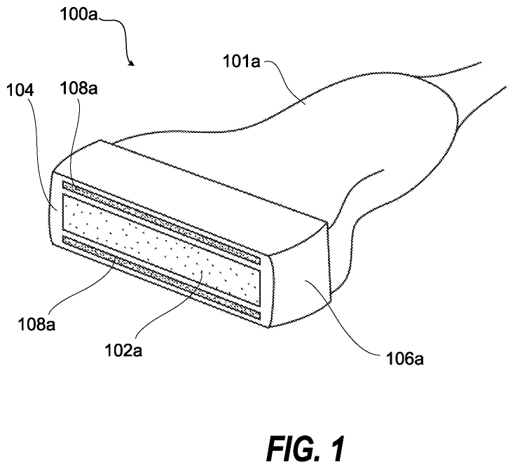

Turning to the Drawing, FIG. 1 is an example of a first probe assembly 100a that includes an ultrasound probe 101a having an ultrasound device 102a along a distal edge 104. In one or more embodiments, a boot 106a, such as molded rubber, attaches via snap-on, friction, straps, or otherwise engagedly attaches (collectively, “snap-on”), around the distal end 104 of the ultrasound device 102a, positioning dual vibratory devices 108a (e.g., dual glass encased piezo-electric bar materials configured for full range audio frequencies as a two-channel attachment) along opposite elongate sides of the ultrasound device 102a. The first probe assembly 100a is shown in FIG. 1 with a strap-on attachment of the piezo-electric bar materials (vibratory devices 108a) enables elastographic and viscoelastographic ultrasound measurements of subject tissue. The dual piezo-electric bars enable a mono or stereo field of vibration to be injected into tissue, producing a number of tactile acoustic wave fields, including, but not limited to, compression/longitudinal wave fields and various shear wave fields (crawling, reverberant, monodirectional, etc.). This method provides a simple and efficient method of transferring non-invasive tactile acoustic vibrations into the tissue for EV-SWEI. Not shown in this figure is the damping material to acoustically isolate the vibrations of the dual piezo-electric bars from the ultrasound device 102a, thereby minimizing the impact of the vibrations on ultrasonic tissue motion tracking.

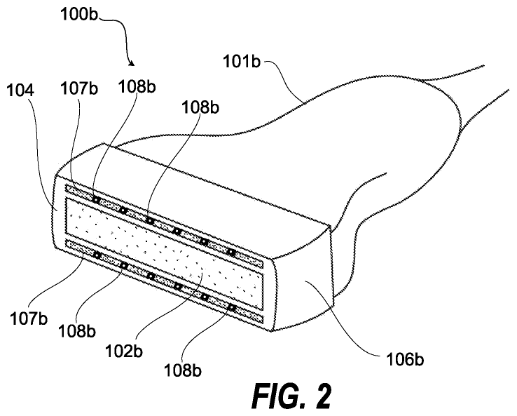

FIG. 2 is an example of a second probe assembly 100b that includes an ultrasound probe 101b having an ultrasound array device 102b along a distal edge 104. As shown in FIG. 1, a molded rubber boot 106b is attached around the distal edge 104 of the ultrasound array device 102b, positioning dual vibratory array devices 107b (in this embodiment, dual segmented glass-encased piezo-electric bar materials configured for full range audio as a two-channel attachment) along opposite elongate sides of the ultrasound device 102b. Each vibratory array device 107b includes multiple vibratory piezo bars 108b. Second probe assembly 100b provides snap-on attachment 106b enables elastographic and viscoelastographic ultrasound vibrations, in which multiple short strips (“segments”) of piezoelectric bar materials are placed in parallel to the ultrasound probe, and in some embodiments on each end of the probe perpendicular to the probe (not shown), and damping material (not shown) to acoustically isolate the vibrations of the dual segmented piezo-electric bars 108b from the ultrasound device 102. This method enables a stereo or multi-channel field of vibration to be injected into tissue, producing a number of tactile acoustic waveforms, including, but not limited to, compression/longitudinal, shear waves (e.g., crawling, reverberant, etc.). This method provides additional benefits such as beam steering, phased alignments, ambisonics, etc., for implementing EV-SWEI methods. For embodiments in which the piezo materials and electronics are selected to enable ultrasonic transmit frequencies and when the number of segments in each bar approximates the number of ultrasound array elements, then ARFI push pulses can also be generated. Note that for both EV-SWEI and ARFI capable embodiments, the ultrasound probe can function entirely within its typical Doppler tracking method and does not need to switch to produce ARFI focused push pulses.

As used herein, the term “damping material” or “dampening material” means a vibration dampening elastic polymer or elastomer material (e.g., a viscoelastic urethane polymer material such as Sorbothane® material manufactured by Sorbothane, Inc., Kent, Ohio), a thermoplastic elastomer material (TPE), a thermoplastic polyurethane material (TPU), and/or other suitable types of materials to absorb shock, isolate vibration, and/or dampen noise. In one or more embodiments, the dampening material comprises one or more polymers selected from the group consisting of polydimethylsiloxane (PDMS), polyvinyl chloride (PVC), ethylene propylene rubber (EPR), styrene butadiene rubber (SBR), nitrile butadiene rubber (NBR), thermoplastic polyurethane (TPU), polyisoprene (IR), PTFE (Teflon), polyethylene, nylon, polyetheretherketone (PEEK), nylon, acrylic (PMMA), polycarbonate (Lexan), polyimide, latex, polyvinylchloride (PVC), silicone rubber, polyurethane and polyesters. In one embodiment, the dampening material is an elastomer such as polyurethane or silicone.

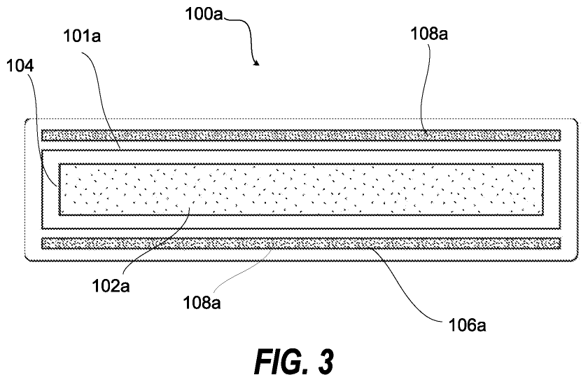

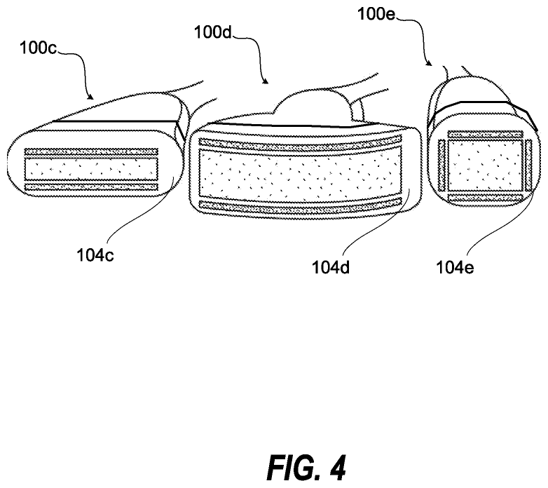

FIG. 3 is a front view of a distal end of the first probe assembly 100a having a planar rectangular alignment of dual vibratory devices 108a on each side of ultrasound device 102a. Second probe assembly 100b (FIG. 2) has a similar or identical planar rectangular alignment. Aspects of the present disclosure may be aligned to other alignments. FIG. 4 depicts three-dimensional views of a respective distal end of three examples of probe assemblies 100c, 100d, and 100e that, rather than planar rectangular, are respectively linear, curvilinear, and phased array distal ends 104c, 104d, and 104e for those respective ultrasound probe types.

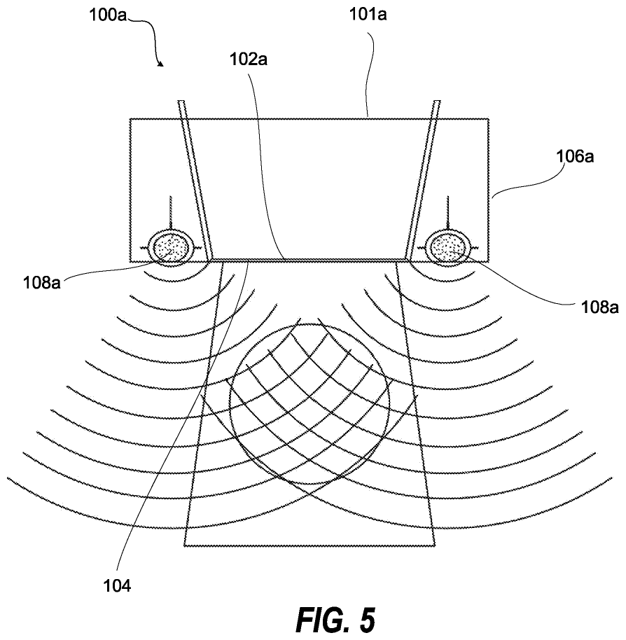

FIG. 5 is an illustration of how two tactile acoustic vibration devices can create a complex overlapping vibration field (indicated by a circle) using the method described in FIG. 1. As depicted, ultrasound device 102a of ultrasound probe 101a is surrounded on left and right sides by molded boot 106a that supports dual vibratory 108a of piezo material within piezo casing. The mono or stereo vibrations are projected from piezoelectric tactile acoustic strips mounted on either side of the ultrasound probe and array and produce several types of waveforms, with shear waves being primarily useful for elastography and viscoelastography. The ultrasound device 102a performs its tracking function using standard methods, e.g., by isonifying the tissue within its transmission angle with ultrasound transmits (typically Doppler chirps) indicated by a trapezoid, recording the received echoes, and then communicating this data to the ultrasound machine for processing and reconstruction.

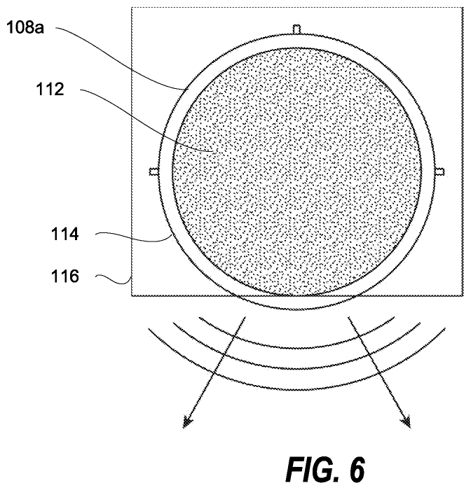

FIG. 6 is a side-view cut away of the vibratory device 108a comprising the piezo bar 112, encased in the piezo casing 114 of glass or hard plastic, surrounded by damping material 116 to acoustically isolate it from the ultrasound device 102a. Optionally, the piezo bar 112, encased in the piezo casing 114 may include spacing or other non-isolating material between the piezo casing 114 and the damping material 116 (not shown).

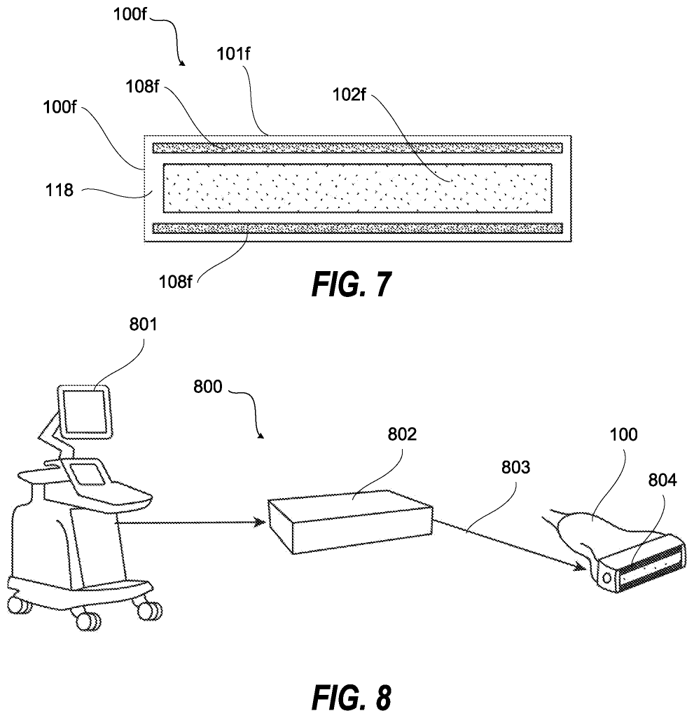

FIG. 7 is an illustration of a probe assembly 100f formed with dual vibratory devices 108f of piezo bar materials and damping materials are embedded directly into the ultrasound probe face 118 that also supports an ultrasound array device 102f, and not as a snap-on attachment. This method would provide an isolated piezoelectric solution that is manufactured as part of an ultrasound probe 101f. This method would include all the components described for the snap-on embodiments, but they would be manufactured as part of the commercial probe. The ultrasound probe face 118 includes or comprises a dampening material around the piezo material to prevent vibration from the piezo materials to be detected by the ultrasound array device 102f.

FIG. 8 is a communication flow chart of an electronics system 800 needed for this method. An ultrasound machine 801 will send waveforms from the various scanning programs to a mono, stereo or multichannel power amplifier 802. Individual line feeds 803 will go from the amplifier 802 to each individual piece of piezoelectric tactile acoustic material 804 of a probe assembly 100, such as one of the previously described probe assemblies 100a, 100b, 100c, 100d, 100e, and 100f. Each piece will require its own feed to allow for stereo or multi-channel applications.

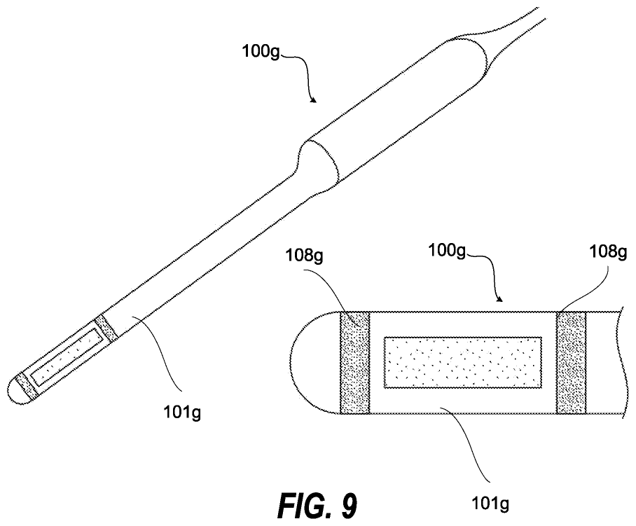

FIG. 9 is three-dimension view with a close-up distal end view of an example probe assembly 100g of two tissue contacting piezo bars 108g in the form of rings embedded into a transrectal ultrasound probe 101g, whereby the piezo rings provide a mono or stereo field of vibration field for use with elastography and viscoelastography medical imaging methods, such as reverberant, crawling wave, and others. Transrectal probes are used for examining abnormalities in the rectum and nearby structures, including the prostate. Also called endorectal ultrasound (ERUS) and transrectal ultrasound (TRUS). Transrectal probes are most commonly inserted into the rectum to examine the prostate. Embedding piezo audio-frequency vibration divers into the transrectal probe allows for the entire process of elastography to be done at the point of contact, within the body, for example, for prostate cancer diagnosis.

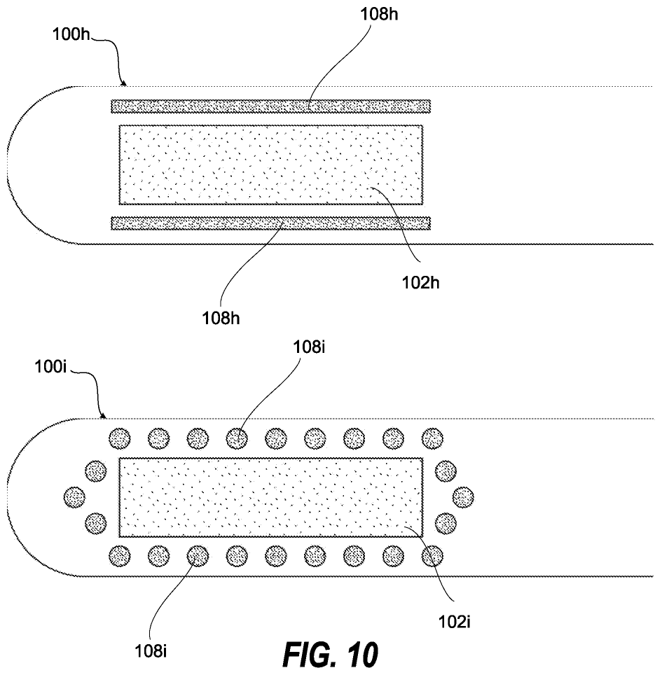

FIG. 10 shows side view of two examples probe assemblies 100h and 100i of alternative piezo bar transducers embedded in a transrectal probe. Probe assembly 100h shows vibratory device 108h of two parallel bars of tissue contacting piezo material aligned in parallel with the ultrasound array 102h. Probe assembly 100i shows vibratory devices 108i of a series of piezo tissue contact “buttons” (small circular piezo parts) surrounding the ultrasound array 102i. This method will allow for multi-source/multi-channel piezo bar sources and can be used for elastography methods such as reverberant elastography and viscoelastography, whereby a multitude of vibration sources can be used to produce multidirectional shear wave fields along with other useful types of shear wave fields.

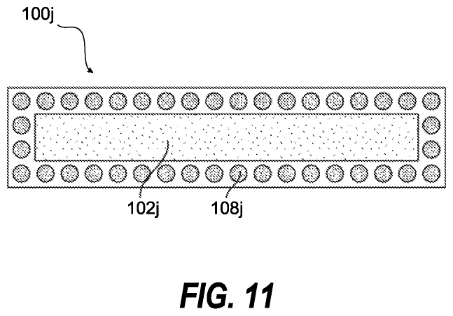

FIG. 11 is an example of a linear ultrasound probe 100j (not transrectal) with vibratory devices 108j of a series of piezo “buttons” (small piezo parts, often circular, surrounding the ultrasound array 102j). This method will allow for multi-source/multi-channel piezo bar sources and can be used to generate a variety of shear wave fields including multidirectional shear wave fields such as reverberant and crawling wave, among others. When the number of piezo buttons substantially matches the number of ultrasound elements, this method can also be used for phased-array techniques of focused vibration for elastography methods such as Acoustic Radiation Force Impulse (ARFI) and its variants such as supersonic imaging (SSI).

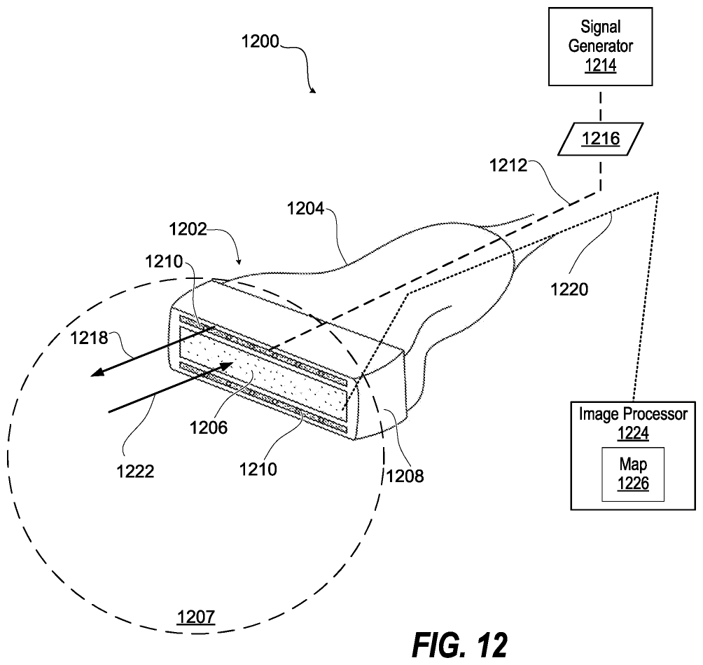

FIG. 12 is a block diagram of an ultrasound elastography diagnostic apparatus 1200 that includes a probe assembly 1202 that includes an ultrasound probe 1204 having an ultrasound device or array of devices 1206 configured to detect vibrations passing through subject tissue 1207. The probe assembly 1202 includes a snap-on molded boot component 1208. The snap-on boot component 1208 includes one or more vibratory devices 1210 and vibration damping material to isolate the ultrasound device 1206 from the vibrations induced by 1210. The probe assembly 1202 includes an input electrical interface 1212 communicatively couplable to a signal generator 1214 to receive a vibration driver signal 1216 comprising one of: (i) an acoustic radiation force impulse (ARFI) signal; and (ii) an external vibration shear wave elastography imaging (EV-SWEI) signal. The input electrical interface 1212 is communicatively coupled to the one or more vibratory devices 1210 to generate a vibration 1218 of one of a corresponding one of an ARFI push pulse and an EV-SWEI vibration. The probe assembly 1202 includes an output electrical interface 1220 communicatively coupled to the ultrasound probe 1204 that receives tissue echoes 1222 from the subject tissue 1207 after each ultrasound transmit for tracking tissue displacement. The output electrical interface 1220 is communicatively couplable to an image processor 1224 to process the tissue echoes into tissue displacements and then into one or more viscoelastic properties that can be displayed as one or more maps 1226 on a display.

In one or more embodiments, the one or more vibratory devices 1210 include one or more piezoelectric bars. In one or more embodiments, 1208 is an overmolded boot that attaches to the ultrasound probe 1204. In one or more embodiments, the ultrasound elastography diagnostic apparatus 1200 is not only communicatively coupled to, but also includes the signal generator 1214 and the image processor 1224. In one or more embodiments, the signal generator 1214 is configured to generate the ARFI signal. The image processor 1224 is configured to map the one or more viscoelastic properties derived from the received tissue echoes 1222 that results from ARFI signal. In one or more embodiments, the signal generator 1214 is configured to generate the EV-SWEI signal. The image processor 1224 is configured to map the one or more viscoelastic properties derived from the received tissue echoes 1222 that results from the EV-SWEI signal.

In one or more embodiments, the imaging processor 1224 is configured to determine the one or more viscoelastic properties of a body region of a living subject at a plurality of points within that body region by: (i) establishing a shear wave field within the body region; (ii) measuring a characteristic of the shear wave field at each of the plurality of points; (iii) computing, at each of the plurality of points, a rate of change of the characteristic with respect to positional change within the body region; and (iv) determining, from the computed rate of change at each of the plurality of points, one or more viscoelastic properties of the body region at the plurality of points. In one or more particular embodiments, the imaging processor 1224 is further configured to establish using at least one vibration source to create a field of shear waves within the body region. In one or more specific embodiments, the one or more viscoelastic properties comprises at least one of stiffness, dispersion and viscosity. In one or more specific embodiments, the signal generator 1214 generates the vibration signal that is configured to subject the body region to shear-inducing vibration delivered at a plurality of frequencies.

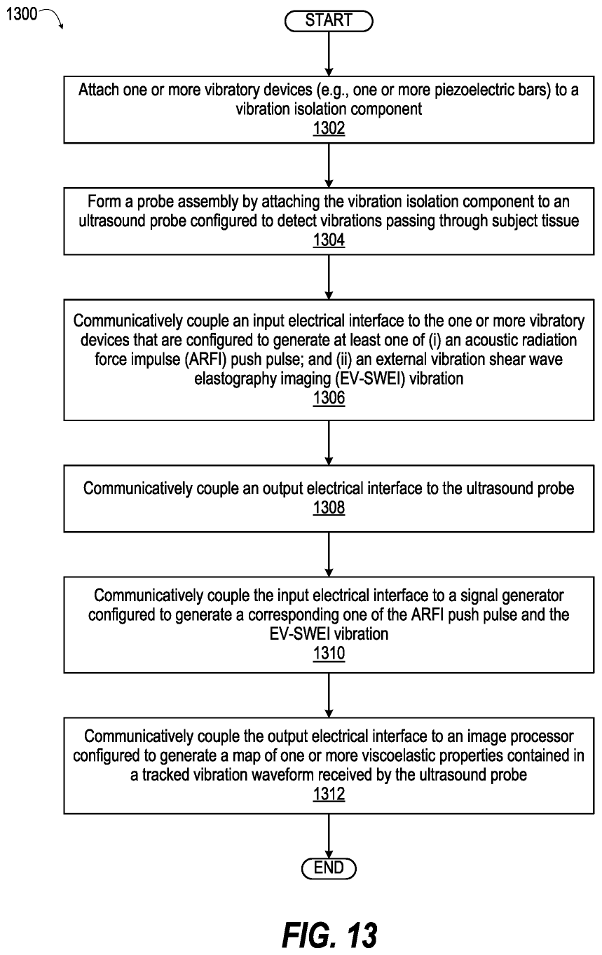

FIG. 13 is a flow diagram of a method 1300 for making an ultrasound elastography diagnostic apparatus. In one or more embodiments, the method 1300 includes attaching one or more vibratory devices (e.g., one or more piezoelectric bars) to a vibration isolation component (block 1302). The method 1300 includes forming a probe assembly by attaching the vibration isolation component to an ultrasound probe configured to detect vibrations passing through subject tissue (block 1304). The method 1300 includes communicatively coupling an input electrical interface to the one or more vibratory devices that are configured to generate at least one of (i) an acoustic radiation force impulse (ARFI) push pulse; and (ii) an external vibration shear wave elastography imaging (EV-SWEI) vibration (block 1306). The method 1300 includes communicatively coupling an output electrical interface to the ultrasound probe (block 1308). The method 1300 includes communicatively coupling the input electrical interface to a signal generator configured to generate a corresponding one of the ARFI push impulse and the EV-SWEI vibration (block 1310). Method 1300 includes communicatively coupling the output electrical interface to an image processor configured to generate a map of one or more viscoelastic properties contained in the information received from the ultrasound probe (block 1312). Then method 1300 ends.

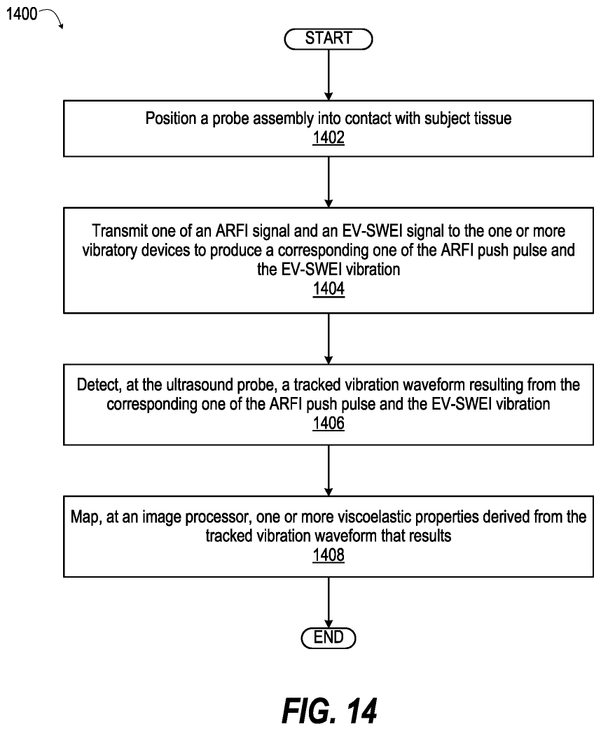

FIG. 14 is a flow diagram of a method 1400 of performing an ultrasound elastography diagnostic procedure. In one or more embodiments, the method 1400 includes positioning a probe assembly into contact with subject tissue (block 1402). The probe assembly includes: (i) an ultrasound probe configured to detect vibrations passing through the subject tissue; (ii) a vibration isolation component; and (iii) one or more vibratory devices coupled to the ultrasound probe via the vibration isolation component. The one or more vibratory devices are configured to generate at least one of (a) an acoustic radiation force impulse (ARFI) push pulse; and (b) an external vibration shear wave elastography imaging (EV-SWEI) vibration. Method 1400 includes transmitting one of an ARFI signal and an EV-SWEI signal to the one or more vibratory devices to produce a corresponding one of the ARFI push pulse and the EV-SWEI vibration (block 1404). Method 1400 includes detecting, using an ultrasound probe, a tracked vibration waveform resulting from the corresponding one of the ARFI push pulse and the EV-SWEI vibration (block 1406). Method 1400 includes mapping, at an image processor, one or more viscoelastic properties derived from the tracked vibration waveform that results (block 1408). Then method 1400 ends.

In one or more embodiments, method 1400 may further include generating the ARFI signal; and mapping the one or more viscoelastic properties derived from the tracked vibration waveform that results from the ARFI signal.

In one or more embodiments, method 1400 may further include generating the EV-SWEI signal; and mapping the one or more viscoelastic properties derived from the tracked vibration waveform that results from the EV-SWEI signal.

In one or more embodiments, method 1400 may further include determining the one or more viscoelastic properties of a body region of a living subject at a plurality of points within that body region by: (i) establishing a shear wave field within the body region; (ii) measuring a characteristic of the shear wave field at each of the plurality of points; (iii) computing, at each of the plurality of points, a rate of change of the characteristic with respect to positional change within the body region; and (iv) determining, from the computed rate of change at each of the plurality of points, one or more viscoelastic properties of the body region at the plurality of points.

In one or more particular embodiments, method 1400 may further include creating a field of shear waves within the body region. In one or more particular embodiments, the one or more viscoelastic properties comprises at least one of stiffness, dispersion and viscosity.

First, by virtue of the present disclosure, in one or more embodiments, an acoustic radiation force impulse (ARFI) ultrasound elastography diagnostic apparatus is provided wherein the two functions of ARFI (push pulse and tracking) are divided into two separate hardware systems, while both remain enclosed in the same apparatus (probe).

Second, in another aspect of the present disclosure, a method is provided of using an acoustic radiation force impulse (ARFI) elastography system wherein the two functions of ARFI (push pulse and tracking) are divided into two separate hardware systems, while both remain enclosed in the same apparatus (probe).

Third, in an additional aspect of the present disclosure, an acoustic radiation force impulse (ARFI) ultrasound elastography diagnostic apparatus is provided wherein the two functions of ARFI (push pulse and tracking) are divided into two separate hardware systems, wherein the tracking function hardware is a standard ultrasound diagnostic probe and the ARFI push pulse hardware is a molded boot that fits onto the standard ultrasound diagnostic probe so that both functions are joined together for operation.

Fourth, in a further aspect of the present disclosure, a method of using an acoustic radiation force impulse (ARFI) elastography system is provided wherein the two functions of ARFI (push pulse and tracking) are divided into two separate hardware systems, wherein the tracking function hardware is a standard ultrasound diagnostic probe and the ARFI push pulse hardware is embedded into a molded boot that fits onto the standard ultrasound diagnostic probe so that both functions are joined together for operation.

Fifth, in yet another aspect of the present disclosure, an external vibration ultrasound elastography apparatus is provided wherein the external vibration system is integral to the ultrasound probe.

Sixth, in yet an additional aspect of the present disclosure, a method of using an external vibration ultrasound elastography apparatus is provided wherein the external vibration system is integral to the ultrasound probe.

Seventh, in yet a further aspect of the present disclosure, an external vibration ultrasound elastography apparatus is provided wherein the external vibration system is embedded into a molded boot that fits onto a standard ultrasound diagnostic probe so that both the external vibrator and ultrasound probe are joined together for operation.

Eighth, in another additional aspect of the present disclosure, a method of using an external vibration ultrasound elastography apparatus is provided wherein the external vibration system is embedded into a molded boot that fits onto a standard ultrasound diagnostic probe so that both the external vibrator and ultrasound probe are joined together for operation.

Ninth, in one or more embodiments of the second, fourth or sixth aspect above, the method includes determining a viscoelastic property of a body region of a living subject at a plurality of points within that body region, comprising the following steps: (i) establishing a shear wave field within the body region; (ii) measuring a characteristic of the shear wave field at each of said plurality of points; (iii) computing, at each of said plurality of points, a rate of change of said characteristic with respect to positional change within the body region; and (iv) determining, from said computed rate of change at each of said plurality of points, a viscoelastic property of the body region at said plurality of points.

Tenth, in a particular embodiment of the ninth aspect, the establishing step may include using at least one vibration source to create a shear wave field within the body region.

Eleventh, in a specific embodiment of the tenth aspect, the viscoelastic property is stiffness or dispersion.

Twelfth, in a very specific embodiment of the eleventh aspect, the establishing step comprises subjecting the body region to shear-inducing vibration delivered at a plurality of frequencies.

Thirteenth, in an additional further aspect of the present disclosure, a system comprises an apparatus according to the first, third, or fifth aspect above and further comprises an image processor configured to provide a map of one or more viscoelastic properties of each of plural points within the region of interest.

It is further envisioned that the probe according to the present system may be used with other types of endocavity probes. For example, the endoscopic devices for imaging according to the present system may include various device types such as TEE, transnasal, transvaginal, transrectal, endo-cavity (e.g., a transducer with a shaft at the end with ultrasound array that moves the array to touch or come close to a mass that is to be operated on for surgical application, for example, inserted through a natural opening or an opening made by a surgeon. The endoscopic devices for imaging according to the present system may be manually and/or automatically controlled, including manual/automatically control from a remote location, i.e., remote from the location of the procedure, where the controller and associated devices such as display, I/O device, memory, are operationally connected to a local controller or processor, through a network, such as the Internet. Control and other signals including image signals may be transmitted and/or received through any means, wired or wireless, for example.

It should be noted that the various embodiments may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a solid-state drive, optical disk drive, and the like. The storage device may also be other similar means for loading computer programs or other instructions into the computer or processor.

As used herein, the term “computer,” “subsystem” or “module” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein. Instead, the use of “configured to” as used herein denotes structural adaptations or characteristics, and denotes structural requirements of any structure, limitation, or element that is described as being “configured to” perform the task or operation. For example, a controller circuit, processor, or computer that is “configured to” perform a task or operation may be understood as being particularly structured to perform the task or operation (e.g., having one or more programs or instructions stored thereon or used in conjunction therewith tailored or intended to perform the task or operation, and/or having an arrangement of processing circuitry tailored or intended to perform the task or operation). For the purposes of clarity and the avoidance of doubt, a general purpose computer (which may become “configured to” perform the task or operation if appropriately programmed) is not “configured to” perform a task or operation unless or until specifically programmed or structurally modified to perform the task or operation.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

The transmitting step requires transmitting at least one ultrasound signal with sufficient power to permit the signal to travel in the tissue of interest. Typically, the transmitted signal will be reflected off an interface that separates two layers that contain differing amounts of water and biomaterials. Any suitable frequency, as described herein or in the future or known in the art can be used. The frequencies used can be selected for maximum transmission and reflective performance, and lowest noise by recording signals from a tissue at different frequencies. Thus, for a particular tissue, the frequency with the best properties can be selected and a dedicated probe can be constructed using such a frequency. transmitting step is desirably practiced using multiple signals. A plurality of signals can be transmitted and their return signals (“echoes”) from reflective interfaces recorded. Signal averaging will improve the accuracy of the measurements and can be conducted over a relatively short period of time. Generally, multiple signals for signal averaging will be transmitted in less than 1 to 2 seconds and more often in less than 100 to 300 milliseconds and preferably in less than 50 milliseconds. The transmitting step can be optionally practiced using multiple signals over longer lengths of time that would not typically be used for signal averaging. A, B or C scan modes of ultrasound interrogation and recording can be used with the methods and devices of the invention. This invention can be applied to a variety of application sites and medical treatments as described herein, developed in the future or known in the art. This invention also can be used with many different types of suitable probes, systems, and methods relating to ultrasound measurements, and calculations and biological standards, as described herein, developed in the future or known in the art.

Signals received by the detector can be subjected to threshold processing. Typically, threshold processing excludes signals of a predetermined value or range of values. The signal processing can potentially exclude signal either above or below the predetermined threshold value. The predetermined threshold value for a signal can include: 1) predetermined values correlated with, or selected from, anatomical sites and structures (e.g., estimates of actual thicknesses), 2) predetermined values generated from interrogating the tissue under examination (e.g., generating average values for the tissue under examination), and 3) predetermined values generated from interrogating tissues to determine normative values for different tissues, subject populations, medical conditions, etc. (e.g., generating average values from particular anatomical sites or structures using multiple qualified subjects). A system or detector can exclude signals at different levels of signal detection or processing. For instance, signals can be excluded by time gating, electronic filtering, digital filtering, analog filtering, and amplitude gating.

Signals, results of calculations, or signal processing can be displayed on a digital or analog display for the operator or the subject to observe. The display can also include a predetermined display arrangement that includes symbols or illustrative graphics of preselected anatomical features of the interrogated tissue. Results of calculations can then be used in the graphic to display the calculated distances (or other suitable information) associated with the predetermined anatomical features.

While the disclosure has been described with reference to exemplary embodiments, it will be understood by those skilled in the art that various changes may be made, and equivalents may be substituted for elements thereof without departing from the scope of the disclosure. In addition, many modifications may be made to adapt a particular system, device or component thereof to the teachings of the disclosure without departing from the essential scope thereof. Therefore, it is intended that the disclosure is not limited to the particular embodiments disclosed for carrying out this disclosure, but that the disclosure will include all embodiments falling within the scope of the appended claims. Moreover, the use of the terms first, second, etc. do not denote any order or importance, but rather the terms first, second, etc. are used to distinguish one element from another.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

The description of the present disclosure has been presented for purposes of illustration and description but is not intended to be exhaustive or limited to the disclosure in the form disclosed. Many modifications and variations will be apparent to those of ordinary skill in the art without departing from the scope of the disclosure. The described embodiments were chosen and described in order to best explain the principles of the disclosure and the practical application, and to enable others of ordinary skill in the art to understand the disclosure for various embodiments with various modifications as are suited to the particular use contemplated.

What is claimed is:

1. An ultrasound elastography diagnostic apparatus comprising:
a probe assembly comprising:
an ultrasound probe configured to detect vibrations passing through subject tissue;
a vibration isolation component;
one or more vibratory devices coupled to the ultrasound probe via the vibration isolation component;
an input electrical interface communicatively couplable to a signal generator to receive a vibration driver signal comprising one of: (i) an acoustic radiation force impulse (ARFI) signal; and (ii) an external vibration shear wave elastography imaging (EV-SWEI) signal, the input electrical interface communicatively coupled to the one or more vibratory devices to generate a corresponding one of an ARFI push pulse and an EV-SWEI vibration; and
an output electrical interface communicatively coupled to the ultrasound probe that tracks tissue displacements and is communicatively couplable to an image processor to generate a map of one or more viscoelastic properties contained in the tracked tissue displacements.

2. The ultrasound elastography diagnostic apparatus of claim 1, wherein the one or more vibratory devices comprises one or more piezoelectric bars.

3. The ultrasound elastography diagnostic apparatus of claim 1, wherein the vibratory devices and isolation components are combined in an overmolded boot that is then attached to the ultrasound probe.

4. The ultrasound elastography diagnostic apparatus of claim 1, further comprising the signal generator and the image processor.

5. The ultrasound elastography diagnostic apparatus of claim 4, wherein:
the signal generator is configured to generate the ARFI signal; and
the image processor is configured to map the one or more viscoelastic properties derived from the tracked tissue displacements that results from ARFI signal.

6. The ultrasound elastography diagnostic apparatus of claim 4, wherein:
the signal generator is configured to generate the EV-SWEI signal; and
the image processor is configured to map the one or more viscoelastic properties derived from the tracked tissue displacements that results from the EV-SWEI signal.

7. The ultrasound elastography diagnostic apparatus of claim 4, wherein the imaging processor is configured to determine the one or more viscoelastic properties of a body region of a living subject at a plurality of points within that body region by:
establishing a shear wave field within the body region;
measuring a characteristic of the shear wave field at each of the plurality of points;
computing, at each of the plurality of points, a rate of change of the characteristic with respect to positional change within the body region; and
determining, from the computed rate of change at each of the plurality of points, one or more viscoelastic properties of the body region at the plurality of points.

8. The ultrasound elastography diagnostic apparatus of claim 7, wherein the imaging processor is further configured to establish, using at least one vibration source, a field of shear waves within the body region.

9. The ultrasound elastography diagnostic apparatus of claim 8, wherein the one or more viscoelastic properties comprises at least one of stiffness, elasticity, viscosity, attenuation, wave speed, phase angle, and frequency dispersion.

10. The ultrasound elastography diagnostic apparatus of claim 8, wherein the signal generator generates the vibration signal that is configured to subject the body region to shear wave inducing vibrations delivered at a plurality of audio frequencies.

11. A method for making an ultrasound elastography diagnostic apparatus, the method comprising:
attaching one or more vibratory devices to a vibration isolation component;
forming a probe assembly by attaching the vibration isolation component to an ultrasound probe configured to detect vibrations passing through subject tissue;
communicatively coupling an input electrical interface to the one or more vibratory devices that are configured to generate at least one of (i) an acoustic radiation force impulse (ARFI) push pulse; and (ii) an external vibration shear wave elastography imaging (EV-SWEI) vibration; and
communicatively coupling an output electrical interface to the ultrasound probe.

12. The method of claim 11, further comprising:
communicatively coupling the input electrical interface to a signal generator configured to generate a corresponding one of the ARFI push pulse and the EV-SWEI vibration; and
communicatively coupling the output electrical interface to an image processor configured to generate a map of one or more viscoelastic properties from the information contained in the data received by the ultrasound probe.

13. The method of claim 11, wherein the one or more vibratory devices comprises one or more piezoelectric bars.

14. A method of performing an ultrasound elastography diagnostic procedure, the method comprising:
positioning a probe assembly into contact with subject tissue, the probe assembly comprising: (i) an ultrasound probe configured to detect vibrations passing through the subject tissue; (ii) a vibration isolation component; and (iii) one or more vibratory devices coupled to the ultrasound probe via the vibration isolation component and comprising one or more vibratory devices that are configured to generate at least one of (a) an acoustic radiation force impulse (ARFI) push pulse; and (b) an external vibration shear wave elastography imaging (EV-SWEI) vibration;
transmitting one of an ARFI signal and an EV-SWEI signal to the one or more vibratory devices to produce a corresponding one of the ARFI push pulse and the EV-SWEI vibration;
tracking with the ultrasound probe the tissue displacements resulting from the corresponding one of the ARFI push pulse and the EV-SWEI vibration; and
mapping, at an image processor, one or more viscoelastic properties derived from the tracked tissue displacements.

15. The method of claim 14, further comprising:
generating the ARFI signal; and
mapping the one or more viscoelastic properties derived from the tracked tissue displacements that results from the ARFI signal.

16. The method of claim 14, further comprising:
generating the EV-SWEI signal; and
mapping the one or more viscoelastic properties derived from the tracked tissue displacements that results from the EV-SWEI signal.

17. The method of claim 14, further comprising determining the one or more viscoelastic properties of a body region of a living subject at a plurality of points within that body region by:
establishing a shear wave field within the body region;
measuring a characteristic of the shear wave field at each of the plurality of points;
computing, at each of the plurality of points, a rate of change of the characteristic with respect to positional change within the body region; and
determining, from the computed rate of change at each of the plurality of points, one or more viscoelastic properties of the body region at the plurality of points.

18. The method of claim 17, further comprising creating a field of shear waves within the body region.

19. The method of claim 18, wherein the one or more viscoelastic properties comprises at least one of stiffness, elasticity, viscosity, attenuation, wave speed, phase angle, and frequency dispersion.

20. The method of claim 18, further comprising subjecting the body region to shear-inducing vibration delivered at a plurality of frequencies.
